(19) United States
(12) Patent Application Publication
(10) Pub. No.: US 2024/0207071 A1
(43) Pub. Date: Jun. 27, 2024

(54) PROSTHETIC FOOT INSERT

(71) Applicant: OTTOBOCK SE & CO. KGAA, Duderstadt (DE)

(72) Inventors: Kathrin POHL, Duderstadt (DE); Markus SCHNEEGANS, Duderstadt (DE); Sven KALTENBORN, Duderstadt (DE); Georg GEHRMANN, Duderstadt (DE); Klaus-Peter ANHALT, Duderstadt (DE)

(21) Appl. No.: 18/553,246

(22) PCT Filed: Mar. 24, 2022
(86) PCT No.: PCT/EP2022/057807

§ 371 Date: Sep. 29, 2023

(30) Foreign Application Priority Data
Mar. 31, 2021 (DE) 10 2021 108 163.7

(51) Int. Cl. A61F 2/66; A61F 2/50

(52) U.S. Cl. CPC A61F 2/66; A61F 2/5044; A61F 2002/6678

(57) ABSTRACT

The invention relates to a prosthetic foot insert comprising: a proximal connection device (10) for fastening to a lower-leg tube or lower-leg shank; at least one base element (20), which is coupled or connected to the connection device (10) and has a forefoot region (21); the base element (20) has a heel region (22) or is connected to a heel region (22), which is formed from a first material and extends from the forefoot region (21) in the posterior direction: wherein the forefront region (21) is formed from a second material, which has a lower modulus of elasticity than the first material and projects forwards beyond the anterior end of the heel region (22).

The invention relates to a prosthetic foot insert having a proximal connection device for fastening to a lower leg tube or a lower leg socket, having at least one base element which is coupled or connected to the connection device and has a forefoot region, wherein the base element has a heel region or is connected to a heel region, the latter being formed from a first material and extends from the forefoot region in the posterior direction.

Prosthetic foot inserts are part of a prosthetic treatment, for example in the case of lower leg amputations. Prosthetic foot inserts form a distal end section of a prosthesis of a lower extremity, and are for example fastened to a lower leg tube or directly to a lower leg socket. The fastening of the prosthetic foot insert is carried out by means of a proximal connection device, which is part of the prosthetic foot insert. The connection device very often has a pyramid adapter, by means of which a multiplicity of adjustments and alignments of the prosthetic foot insert relative to a proximal component may be performed. The connection device may furthermore have a bearing block, on which further components of the prosthetic foot insert are arranged, fastened or mounted. Furthermore, the prosthetic foot insert often has a sole element, which either comes directly in contact with the ground or a shoe or is enclosed by a prosthetic cosmetic or a cover. The prosthetic cosmetic or the cover are used on the one hand to protect the technical components of the prosthetic foot insert and on the other hand to provide an impression of the finished prosthetic foot which is as natural as possible. In principle, it is also possible to use the prosthetic foot insert without a prosthetic cosmetic.

The sole element may have a contour on the ground side, which is generally curved. In the heel region and the ball region, downwardly directed convex bends are often formed in order to allow rolling after a heel strike and rollover at the end of the stance phase. Contouring may also be carried out over the cover or the prosthetic cosmetic, which generates a corresponding rolling surface of the lower side of the prosthetic foot by means of material selection and thickening or material conversations.

Despite the configuration of the sole element with a contour on the ground side, problems occur with large foot lengths, especially when walking uphill, since rolling of the prosthetic foot becomes difficult. This is because the forefoot lever is very long in the case of a long prosthetic foot, so that during a rolling movement and concomitant forefoot loading, a very large ankle moment which counteracts the rolling movement is generated. In order to overcome this problem, separate toe elements are fastened on a base element in such a way that they can be pivoted about a rotation axis. Such a design of a prosthetic foot is relatively elaborate.

It is an object of the present invention to provide a prosthetic foot insert which, with a high-quality visual appearance, allows good guiding of the prosthetic foot and provides sufficient safety even in particular movement situations and states.

According to the invention, this object is achieved by a prosthetic foot insert having the features of the main. Advantageous configurations and developments of the invention are disclosed in the dependent claims, the description and the figures.

The prosthetic foot insert having a proximal connection device for fastening to a lower leg tube or a lower leg socket, having at least one base element which is coupled or connected to the connection device and has a forefoot region, wherein the base element has a heel region or is connected to a heel region, the latter being formed from a first material and extends from the forefoot region in the posterior direction, provides that the forefoot region is formed from a second material which has a lower modulus of elasticity than the first material and projects forward beyond the anterior end of the heel region. Making the forefoot region from a second material, which has a lower modulus of elasticity than the first material of the base element, generates increased flexibility and elasticity in the forefoot region and locally significantly different yielding of the base element. Because of the increased flexibility and elasticity, the forefoot region or toe region is capable of deforming under forefoot loading, so that a contact area of the prosthetic foot or of the prosthetic foot insert is generated when the prosthetic foot wearer kneels. Furthermore, a large supporting foot length is avoided when a prosthetic foot user is ascending a ramp or walking on rising ground. Making the forefoot region from a softer material facilitates the generation of an overall visual impression which resembles the natural foot, and in particular is similar in respect of foot length, without the otherwise associated problems during rolling having to be tolerated. Furthermore, an extensive bearing area or contact area is provided, which allows improved control during forefoot loading. In addition, when wearing different shoes with heels of different heights, the prosthetic foot insert adapts well to the shoe and the foot setting without comprehensive adjustments having to be carried out.

The construction of the base element from two materials or two material compositions with the softer material in the forefoot region facilitates the design of the prosthetic foot insert since it can be configured and produced as a single component. A mechanically complex construction with a hinge joint and springs or damping devices or an articulatedly mounted toe element is obviated. Material weakening is in the base element, which is often formed as a fiber reinforced material, are likewise obviated.

Particularly in kneeling positions, the yielding configuration of the forefoot region allows an increase in stability since the second material executes a dorsiflexion and a relatively wide contact area is generated. In this way, tilting or swinging on an otherwise rigid, round forefoot edge is avoided.

According to one development, the base element is configured as a guide element, base spring or downwardly inclined forefoot spring. Particularly in a configuration as a guide element or base spring, the base element serves as a sole part which forms the lower distal structure of the prosthetic foot insert. For example, a heel cushion may be arranged on the lower side of the base spring or of the guide element in order to damp the heel strike and facilitate rolling. Further components of the prosthetic foot insert, for example springs and dampers, which establish a connection to the proximal connection device, then extend from the base spring or the guide element in the proximal direction. A guide element provides no energy storage capacity, or only a small energy storage capacity, while a base spring imparts primarily elastic functionalities. In another configuration, the base element is configured as a forefoot spring which extends into the forefoot region and reaches as far as the front end of the prosthetic foot insert. In order to facilitate or permit a heel strike, a heel element or a heel region is arranged, in particular fastened, on such a forefoot spring. This heel element may have resilient and/or damping properties. In order to guide the movement, the guide element, which is supported via other components on the forefoot spring or further components of the prosthetic foot insert and reaches as far as the forefoot region or into the forefoot region, is arranged or fastened on the heel element.

In one configuration, the base element may be coupled to the connection device by means of at least one forefoot spring, at least one heel spring and/or at least one heel cushion. Prosthetic foot inserts have particular damping properties and elastic properties, which are achieved by a combination of various spring elements and damper elements. In order to couple or indirectly connect the connection device to the base element, various elastic components and damping components may be arranged between them. A direct or immediate connection to the connection device is obtained when it is fastened on the base element without the interposition of functional components. The connection device may, for example, have a bearing block that is mounted pivotably via a holder on a forefoot spring, which is in turn supported on the base element. On the heel side, the connection device may be supported on the base element by means of one or more heel cushions, which may be configured as spring-damper devices. The coupling may be carried out by means of one or more straps, screws, rivets, hook and loop fasteners, other form-fit elements or materially by means of welding or adhesive bonding.

According to one development, a reception pocket which is configured to receive a spring component or a further spring component is arranged on the base element. This spring component or this further spring component is supported on this reception pocket or in this reception pocket and allows durable fastening of the further spring component on the base element. The reception pocket is preferably formed from the first material of the base element and correspondingly has a structure which is not very flexible, durable and dimensionally stable. The further spring component may be fastened inside the reception pocket or on the reception pocket in a variety of ways, for example adhesively bonded, welded and/or fastened by means of form-fit elements or fastening devices.

The forefoot region consisting of the second material is, in one development, overmolded, injection-molded or formed by the two-component injection-molding method or by an additive production method on the base element or materially fastened thereon. The forefoot region is therefore not nondestructively releasably fastened on the heel region. The one-piece configuration of the base element from two different materials allows simple handling and easy mounting on the other components of the prosthetic foot insert. The base element may be manufactured as a constituent part for various weight classes with different strategies for the respective material. The first material is, for example, designed to receive loads during the heel strike and has a many times higher stiffness and many times higher modulus of elasticity than the second material. The first material may be selected and processed depending on the level of activity and the weight of the patient. In a configuration of the base element as a guide element for the heel components, a high dimensional stability with a low use of material is reached by the high modulus of elasticity, so that precise guiding is achieved with a low weight. The second material may be arranged on or connected to the first material so that the first material is locally embedded in the second material. For example, a front region of the first material of the base element, to which the forefoot region is adjacent, may be enclosed by the second material, that is to say be arranged on the lower side, the medial and lateral side faces and on the upper side of the base element, and extend from there in the anterior direction beyond the front end of the first material.

The first material may be formed as a fiber reinforced material or as a polyamide (PA), a thermoplastic copolyester (TPC) elastomer or a polyether ether ketone (PEEK), and the second material may be formed as a thermoplastic polyamide (TPA) elastomer, a thermoplastic copolyester (TPC) elastomer, a thermoplastic olefin (TPO) elastomer, a thermoplastic styrene (TPS) block copolymer or a urethane-based thermoplastic (TPU) elastomer.

The first material may have a modulus of elasticity greater by a factor of 2 to 10 than the second material, all values between the upper and lower maximum ranges being suitable for an expedient design of the base element, or of the prosthetic foot insert.

In one development, the forefoot region has a hallux section, a lateral toe section and a recess between them, in order to provide a shape which approximates the natural foot shape. The hallux section forms the medial and anterior closure of the forefoot region and may extend beyond the lateral toe section in the anterior direction. The lateral toe section may be configured as a continuous surface or with individual toe regions or only contoured toe regions. The recess between them, which corresponds to the interdigital space between the hallux and the toes immediately adjacent thereto, is used in particular to receive a sandal thong or a correspondingly shaped region of a prosthetic foot cosmetic. This provides security of the prosthetic foot insert against turning in the prosthetic cosmetic.

In one development, the forefoot region protrudes medially and/or laterally beyond the first material of the base element. The second material of the forefoot region therefore extends medially and lateral next to the front end of the first material and, in comparison with the first material, provides higher flexibility with an increased lateral contact area.

On the lower side of the base element, in one development, a ledge which extends locally or fully over the width of the base element is formed or arranged. The ledge is preferably formed from the first material and defines a rolling contour, by means of which a precise rolling movement may be achieved in the region of the terminal stance phase. The ledge may also be covered fully or partially by the second material and/or adjoin this ledge in the anterior direction. By means of the second material, the behavior of the prosthetic foot insert in the region of the terminal stance phase or in particular situations, such as kneeling down, may be adjusted and adapted to the properties of the rest of the prosthetic foot insert. In one variant, the ledge is formed by the forefoot region, so that a softer and more yielding rolling behavior and a higher flexibility and elasticity are achieved in comparison with formation by the first material.

The ledge may have a round or rounded cross section, in particular with a curvature that decreases in the anterior direction, so that the radius of curvature increases toward the front end of the prosthetic foot insert. In this way, a larger flat region, which extends over a relatively large region in the mediolateral direction, is provided as a bearing area during rolling or when kneeling down. Together with the dorsally flexed toe region and the dorsally flexed hallux, increased stability is provided against tilting in the mediolateral direction, without awkward rolling properties occurring when walking. In one configuration, the ledge forms a rolling edge which bears on the ground over the width of the ledge in the terminal stance phase. The hard material of the ledge and the wide linear contact in the mediolateral direction provide advantageous stability for the user, particularly in the terminal stance phase.

The forefoot region may have an edge and upwardly directed bulges on the lower side, so that uniform contact is achieved on the outer circumference of the forefoot region. The bulges provide a sufficient volume so that the prosthetic foot insert may be configured with different shapes and contours which are approximated to the natural foot shape, despite a uniform wall thickness. In this way, it is not necessary to provide a prosthetic cosmetic having different material thicknesses in order to achieve shaping approximated to the natural foot. The bulges or excavations serve to save material and facilitate manufacture, particularly in an injection-molding method.

In one development, stabilizing struts, with which locally different flexibilities may be adjusted in the forefoot region, are formed in the forefoot region.

Exemplary embodiments of a prosthetic foot insert are explained in more detail below with the aid of the appended figures, in which:

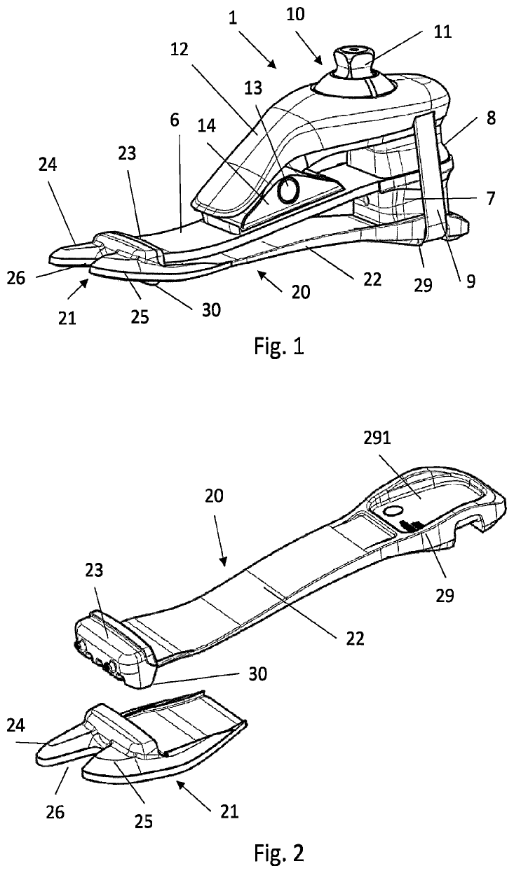

FIG. 1—shows a perspective view of a prosthetic foot insert;

FIG. 2—shows a representation of a base element with separate material sections;

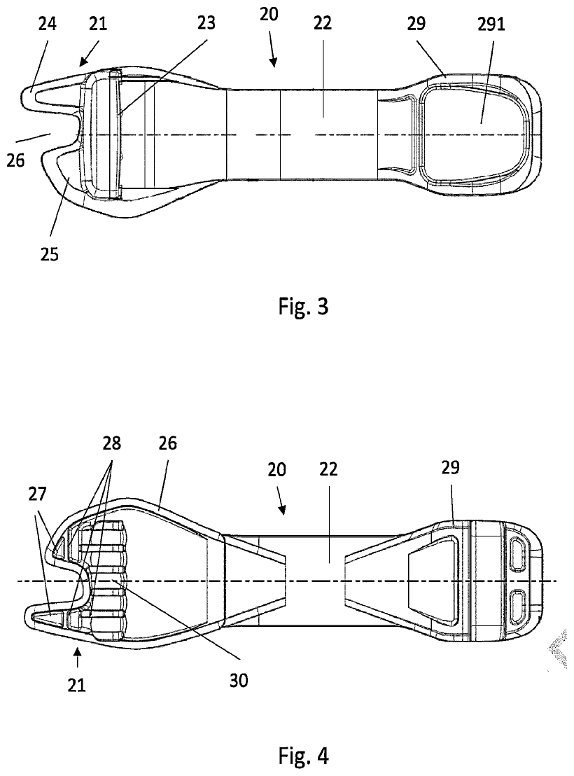

FIG. 3—shows a plan view of the base element;

FIG. 4—shows a view of the base element from below;

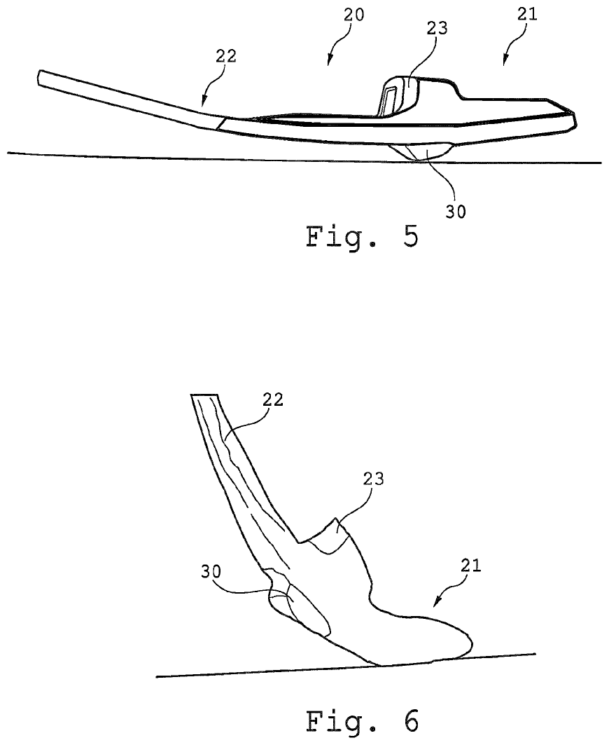

FIG. 5—shows a detail representation in side view; and

FIG. 6—shows a side view in the dorsally flexed state.

FIG. 1 shows a side view of a prosthetic foot insert 1 having a connection device 10 for fastening to a proximal prosthetic component (not represented), for example a lower leg tube or a lower leg socket. The connection device 10 has a pyramid adapter 11, which is fastened or formed on a holder 12. The holder 12 is in particular formed from a dimensionally stable material, for example a light metal, and in the exemplary embodiment is provided with a cladding. Arranged on the front end region of the holder 12 as seen in the walking direction, there is a bearing block 14 which can be tilted about a rotation axle 13 and is connected to a forefoot spring 6. By means of the bearing block 14, it is possible for the holder 12 to be displaced relative to the forefoot spring 6 during loading, which may be necessary in the event of deformations during the loading. The rear end of the holder 12 is supported by means of two heel cushions 8, 7 on the rear end of a base element 20, which in the exemplary embodiment represented is configured as a guide element with little spring action. Alternatively, the base element 20 is configured as a base spring. The rear end of the forefoot spring 6 is arranged between the two heel cushions 7, 8. The forefoot spring 6 either may be held in a clamping fashion between the two heel cushions 7, 8 or is fastened on at least one of the heel cushions 7, 8, for example fastened with a form-fit or force-fit or adhesively bonded.

In order to ensure association of the connection device 10 with the base element 20 in the rear, posterior section of the latter, a strap 9 is passed around the upper side of the holder 12, below the cladding and below the base element 20, and is adjusted in its length so that the heel cushions 7, 8 are prestressed. The forefoot spring 6 is thereby clamped and held between the two heel cushions 7, 8.

In the region of the front end of the forefoot spring 6, the reception pocket 23 is arranged on the base element 20, for example having a slot into which the front end of the forefoot spring 6 is inserted and optionally secured by a fastening element or by adhesive bonding or welding. The reception pocket 23 forms an abutment, so that the forefoot spring 6 cannot be displaced forward beyond the base element 20. As an alternative to a material connection, the forefoot spring 6 is coupled to the base element 20 in such a way that it can be released without the base element 20, the reception pocket 23 and/or the forefoot spring 6 being destroyed. In this way, it is possible to replace the base element 20 and to customize the prosthetic foot insert 1.

The base element 20 has a forefoot region 21 at the front end in the walking direction, which is positioned in the walking direction before the first end of the forefoot spring 6 and before the reception pocket 23. Arranged or formed on the rear end of the base element 20 in the walking direction, there is a fastening region 29 which has form-fit elements in order to fasten a rear force transmission element or a ledge thereon and to secure the strap 9 against accidental displacement in the anterior or posterior direction. As an alternative thereto, the respective force transmission element is fixed on the fastening region 29 with a force-fit, for example held in a clamping fashion or by means of a magnetic retainer. As an alternative or in addition, fixing by means of a clip connection, hook and loop fasteners or fastening by means of separate fastening elements such as pins, springs or screws are likewise possible and provided. Formed on the upper side of the fastening region 29, there is a reception device 291 for receiving the lower heel cushion 7, into which the heel cushion 7 can be inserted. The heel cushion 7 is thereby secured against lateral displacement and against displacement in and counter to the walking direction. The strap 9 and the prestress prevent the heel cushions 7, 8 from being able to loosen in the proximal direction.

On the lower side, approximately at the height of the reception pocket 23, the prosthetic foot insert 1 has a ledge 30 so that a substantially linear force transmission region is formed in the event of forefoot loading. The load transmission during the loading of the forefoot region 21 takes place relatively far to the front as seen in the walking direction, approximately at the height of the metatarsophalangeal joints in the case of a natural foot, so that the maximum lever length for bending moment transmission into the prosthetic foot insert 1 is achieved. By the substantially linear force transmission region, it is possible to absorb and release a very high energy density so that a high dynamic range may be achieved during walking. Because of the force transmission region positioned far to the front and the linear contact with a small contact area, increased flexibility is provided when standing. The ledge 30 ends approximately at the height of the natural metatarsophalangeal joint, which in purely technical terms would be sufficient to ensure the essential functions of walking for the prosthetic foot insert 1. The overall visual impression as well as the psychological effect of a foot shorter by from 20 to 25% is disadvantageous, however, and corresponding devices for fixing the prosthetic foot insert 1 in a prosthetic cosmetic need to be found and arranged.

The base element 20 has a base spring or guide element has a heel region 22, which extends from the reception pocket 23 to a rear, posterior end. The material of the heel region 22 has a relatively high modulus of elasticity in order to absorb the forces incurred at the moment after a heel strike, during the rolling phase and during increasing forefoot loading, or in order to ensure stable and precise guiding of the movement of the heel cushions 7, 8. During a normal step, for example on the flat, after the heel contact and the planting of the entire foot, the essential force transmission takes place at the rear end of the heel region 22 and on the lower ledge 30, and possibly a region adjacent thereto rearward in the walking direction. In a similar way to a natural foot, the heel region 22 has an upwardly directed curvature in the central foot region, so that no ground contact or only slight round contact exists during the stance phase in the region below the articulation axle 13. The forefoot region 21, which consists of a softer material than the material of the heel region 22, extends forward beyond the heel region 22. In other words, the material of the forefoot region 21 has a lower modulus of elasticity than the material of the heel region 22. The material of the heel region 22 is for example a polyamide, although fiber reinforced materials or materials with comparable mechanical properties may alternatively be used. Materials which may be used for a 2-component injection-molding method are advantageous since the possibility of injection-molding the forefoot region 21 then exists. The forefoot region 21 may also be connected durably and nondestructively releasably to the material of the heel region 22 in other ways. It is likewise possible for the base element 20 to be produced in the scope of an additive manufacturing method.

Particularly in situations or states in which a very pronounced dorsiflexion of the forefoot region 21 occurs, for example when walking uphill, when ascending ramps or when kneeling, making the forefoot region 21 from a soft material has substantial advantages. Although, besides shaping which is approximated to the natural foot shape, the softer material of the forefoot region 21, for example a TPU having a modulus of elasticity of between 5% and 50% of the modulus of elasticity of the first material, offers only a lower resistance against bending, it does increase the contact area and ensure stability against sideways tilting in the medial direction or lateral direction. In particular, the yielding of the forefoot region 21 in a configuration with a recess 26 between a hallux section 24 and a lateral toe section 25 prevents the prosthetic foot having to be planted on the tip of the hallux section 24 and tilting about a point contact.

FIG. 2 shows the base element 20 in the manner of an exploded representation, in which the different material regions, which are fastened to one another nondestructively releasably, are shown separated from one another. The heel region 22 adjoins the reception pocket 23 and the ledge 30 arranged underneath. The reception pocket 23 and the ledge 30 are formed from the same material as is used for the heel region 22. Because of the high modulus of elasticity of the material, a high deformation resistance is ensured. The heel region 22 does not need to extend as far as the rear end of the base element 22, although it does in an advantageous configuration. The fastening region 29 may be overmolded on the rear, posterior end of the base element 20. It is also possible for the rear part of the base element 20 to have a different stiffness or a different modulus of elasticity than the anterior or central region. The material thickness of the base element 20 may be different over the longitudinal extent of the latter. The forefoot region 21 is represented separated from the heel region 22 of the base element 20. The forefoot region 21 has a material section extending in the walking direction behind the reception pocket 23, or behind the ledge 30, which is used to protect the lower side of the hard or stiff guide of the heel region 22. FIG. 2 furthermore shows that the softer material of the forefoot region 21 is arranged medially and laterally with respect to the heel region 22, so as to achieve broadening of the contact area and improved stability in the mediolateral direction. Furthermore, this lateral broadening better replicates the natural foot shape.

On the upper side of the forefoot region 2, the material is curved in order to replicate the spatial appearance of a natural toe or the toes. The upper side of the reception pocket 23 is covered at least partially by the softer material of the forefoot region 21 formed integrally with the heel region 22 adjacent in the posterior direction.

FIG. 3 shows the finished base element 20 in a plan view. The relatively narrow, rectilinearly configured heel region 22 is arranged between the fastening region 29 and the forefoot region 21. A slight broadening in the front region in the direction of the reception pocket 23 increases the tilting stability. The broadening in the fastening region 29 with the reception 291 may also be seen as a cup or border for fixing the lower cushion element 7 in five directions.

FIG. 4 shows the base element 20 according to FIG. 3 from the lower side. Besides the fastening region 29 on the rear end of the heel region 22 with a corresponding oblique or rounded lower contour to facilitate rolling and the groove to receive the strap 9, the overmolding of the forefoot region 21 on the front region of the base element 20 may be seen. The forefoot region 21 has a circumferential edge 26 on the lower side, substantially running in a plane, from which the bulges 27 extend upward in order to form the upper-side contour or shape in the toe section 25, or the hallux section 24. Stabilizing struts 28 are overmolded inside the bulges 27 in order to achieve a desired stability inside these regions. Likewise visible on the lower side is the ledge 30, which extends over the entire width of the heel region 22 and is also covered on the lower side by the softer material of the forefoot region 21.

FIG. 5 shows a lateral detail view of the front region of the base element 20 with the heel region 22 and the reception pocket 23 formed integrally with the heel region 22, and the overmolded ledge 30 ledge likewise formed integrally with the heel region 22. In this exemplary embodiment, neither the reception pocket 23 nor the ledge 30 is fully encapsulated with the soft second material of the forefoot region 21. On the reception pocket 23, the rear region in the walking direction is left free, and the side faces on the ledge 30 are not coated with the softer material. The forefoot region 21 protrudes forward in the anterior direction beyond the anterior end of the reception pocket 23 and of the ledge 30. The reception pocket 23 and the ledge 30 form the anterior front end of the heel region 22.

FIG. 6 shows the base element 20 according to FIG. 5 in a position such as is reached for example at the end of the rolling phase, when walking uphill or in a kneeling position. The forefoot region 21 is dorsally flexed and provides a flat contact for the prosthetic foot insert 1. When the ledge 30, which has a rounded, forwardly flattened contour, is likewise resting on the ground, there is linear contact in the region of the ledge 30 and an adjacent flat contact, which is provided by the softer material of the forefoot region 21.

By the front end overmolded, injection-molded around or on, or integrally produced by the forefoot region 21 from a relatively soft material, a visually large foot length with a correspondingly good aesthetic effect is achieved, the forefoot region 21 being flexible and generating a wide contact area of the prosthetic foot when the prosthesis wearer kneels. Such a contact area is generated by additional medial and/or lateral overmoldings or regions of the softer material next to the reception pocket 23 and optionally next to the ledge 30. Despite the visually appealing large effective foot lengths, an excessive effective foot length is avoided in particular walking situations and positions. By the forefoot region 21, a function approximated to the natural foot function may be achieved without great design outlay. Furthermore, noise during the movement is prevented and the risk of mechanical failure is reduced.

The heel region 22 is used as the major functional component for receiving the mechanical loads and is formed, for example, as a polyamide with a shear stress of 50 MPa. The forefoot region with the softer material, for example a technical polyurethane, may for example have a shear stress of 5 MPa.

1. A prosthetic foot insert, comprising:
a proximal connection device for fastening to a lower leg tube or a lower leg socket;
at least one base element is coupled or connected to the connection device; and
a forefoot region,
wherein the at least one base element has a heel region or is connected to the heel region, wherein the heel region is formed from a first material and extends from the forefoot region in a posterior direction,
wherein the forefoot region is formed from a second material which has a lower modulus of elasticity than the first material and projects forward beyond an anterior end of the heel region.

2. The prosthetic foot insert as claimed in claim 1, wherein the at least one base element is configured as a guide element, a base spring, or a downwardly inclined forefoot spring.

3. The prosthetic foot insert as claimed in claim 1 further comprising a reception pocket for a spring component formed on the at least one base element from the first material.

4. The prosthetic foot insert as claimed in claim 1 wherein the forefoot region is overmolded, injection-molded, formed by a two-component injection-molding method, or formed by an additive production method on the at least one base element, or wherein the forefoot region is materially fastened on the at least one base element.

5. The prosthetic foot insert as claimed in claim 1 wherein the first material is a polyamide, a thermoplastic copolyester elastomer, or a polyether ether ketone, and wherein the second material is a thermoplastic polyamide elastomer, a thermoplastic copolyester elastomer, a thermoplastic olefin (TPO) elastomer, a thermoplastic styrene (TPS) block copolymer, or a urethane-based thermoplastic (TPU) elastomer.

6. The prosthetic foot insert as claimed in claim 1 wherein the first material has a modulus of elasticity greater than the second material by a factor of 2 to 10 than the second material.

7. The prosthetic foot insert as claimed in claim 1 wherein the forefoot region comprises a hallux section, a lateral toe section, and a recess between the hallux section and the lateral toe section.

8. The prosthetic foot insert as claimed in claim 1 wherein the forefoot region protrudes medially and/or laterally beyond the first material of the at least one base element.

9. The prosthetic foot insert as claimed in claim 1 further comprising a ledge which extends locally or fully over a width of the at least one base element, wherein the ledge is formed or arranged on a lower side of the at least one base element.

10. The prosthetic foot insert as claimed in claim 9, wherein the ledge is formed on the at least one base element or by the forefoot region.

11. The prosthetic foot insert as claimed in claim 9 wherein the ledge has a round or rounded cross section provided with a curvature that decreases in an anterior direction.

12. The prosthetic foot insert as claimed in claim 9 wherein the ledge forms a rolling edge which bears on ground over a width of the ledge in a terminal stance phase.

13. The prosthetic foot insert as claimed in claim 1 wherein the forefoot region comprises an edge and upwardly directed bulges on a lower side.

14. The prosthetic foot insert as claimed in claim 1 further comprising stabilizing struts formed in the forefoot region.
